(19) United States
(12) Patent Application Publication
(10) Pub. No.: US 2006/0106269 A1
(43) Pub. Date: May 18, 2006

(54) Methods for designing and operating a dehydrogenation process system that uses a high stability dehydrogenation catalyst

(75) Inventors: Robert Dielman Culp, Katy, TX (US); Richard Douglas Newman, Houston, TX (US)

Correspondence Address: Shell Oil Company, 910 Louisiana, Houston, TX, 77002, US

(73) Assignee: Shell Oil Company

(21) Appl. No.: 11/280,747

(22) Filed: Nov. 16, 2005

Related U.S. Application Data
(60) Provisional application No. 60/629,267, filed on Nov. 18, 2004.

(51) Int. Cl. C07C 2/64

(52) U.S. Cl. 585/444

(57) ABSTRACT

A method of improving the operation a dehydrogenation reactor system having a dehydrogenation reactor defining a dehydrogenation reaction zone and containing a first volume of a dehydrogenation catalyst. The method comprises removing from the dehydrogenation reactor at least a portion of the first volume of the dehydrogenation catalyst; placing in the dehydrogenation reactor having removed therefrom the at least a portion of the first volume a second volume of a high stability dehydrogenation catalyst to thereby provide a second dehydrogenation reactor system; operating the second dehydrogenation reactor system under a dehydrogenation reaction condition; and controlling the dehydrogenation reaction condition so as to provide a desired deactivation rate of the high stability dehydrogenation catalyst.

This application claims the benefit of U.S. Provisional Application No. 60/629,267 filed Nov. 18, 2004, the entire disclosure of which is hereby incorporated by reference.

The invention relates to the design and operation of a dehydrogenation process system that uses a high stability dehydrogenation catalyst.

In the field of catalytic dehydrogenation of alkylaromatic hydrocarbons to alkenylaromatic hydrocarbons there are ongoing efforts to develop improved catalysts that have the properties of high activity and selectivity while exhibiting high stability when in use. The stability of a catalyst refers to its rate of catalytic deactivation or decline when in use. The rate of deactivation of a catalyst impacts its useful life, and, generally, it is preferable for a catalyst to be highly stable so as to increase its life and to provide other benefits.

The stability of a dehydrogenation catalyst used in a process for manufacturing styrene by the dehydrogenation of ethylbenzene can have an impact on the operation of such a process. For example, typically the process starts operation with a fresh load of dehydrogenation catalyst that provides for a certain ethylbenzene conversion at a start-of-run reaction temperature. As the process is operated over a time period, the dehydrogenation catalyst will tend to deactivate thereby resulting in a higher reaction temperature required to achieve the same certain ethylbenzene conversion. With time, the reaction temperature will continue to be increased to offset the effects of catalyst deactivation until the temperature reaches a level that is not sustainable due to either equipment or economic limitations. When the process reaches this end-of-run reaction temperature condition, the reactor is shut down with the dehydrogenation catalyst being removed and replaced. The procedure for shutdown and catalyst replacement may take up to two to four weeks to complete.

The use of more stable dehydrogenation catalysts in dehydrogenation processes can provide numerous advantages. In existing dehydrogenation plants the more stable catalysts can provide, for example, for longer run lengths, or if a longer run length is not desired, the more stable catalyst may be used to provide for higher conversions by operating under more severe reactor temperature conditions so as to provide for deactivation rates that are similar to those provided by less stable catalysts. Also, the more stable catalysts can provide for greater flexibility in the design of new dehydrogenation process facilities.

With the growing availability of high stability dehydrogenation catalysts it is desirable to be able take advantage of their properties in the operation of dehydrogenation processes or in the design of new dehydrogenation processes.

It is, thus, an object of the invention to provide a method of improving the operation of a dehydrogenation reactor system utilizing a high stability dehydrogenation catalyst.

Another object of the invention is to provide a method that considers the properties of a high stability dehydrogenation catalyst in the design of a dehydrogenation reactor system.

Accordingly, one of the inventions is a method of improving the operation a dehydrogenation reactor system having a dehydrogenation reactor defining a dehydrogenation reaction zone and containing a first volume of a dehydrogenation catalyst. The method comprises removing from the dehydrogenation reactor at least a portion of the first volume of the dehydrogenation catalyst; placing in the dehydrogenation reactor having removed therefrom the at least a portion of the first volume a second volume of a high stability dehydrogenation catalyst to thereby provide a second dehydrogenation reactor system; operating the second dehydrogenation reactor system under a dehydrogenation reaction condition; and controlling the dehydrogenation reaction condition so as to provide a desired deactivation rate of the high stability dehydrogenation catalyst.

Another of the inventive methods includes the design of a dehydrogenation reactor system, which includes a reactor that defines a reaction zone and contains a volume of a high stability dehydrogenation catalyst, wherein the high stability dehydrogenation catalyst is characterized by a catalyst stability property function. The design method comprises selecting a desired run length for the dehydrogenation reactor system; using the catalyst stability property function to determine a standard reactor operating condition required to provide the desired run length; and using the standard reactor operating condition to determine a reactor volume for the reactor required to provide the desired run length. After the dehydrogenation reactor system is designed, the provided dehydrogenation process system is equipped with the reactor having the reactor volume and containing the volume of the high stability dehydrogenation catalyst.

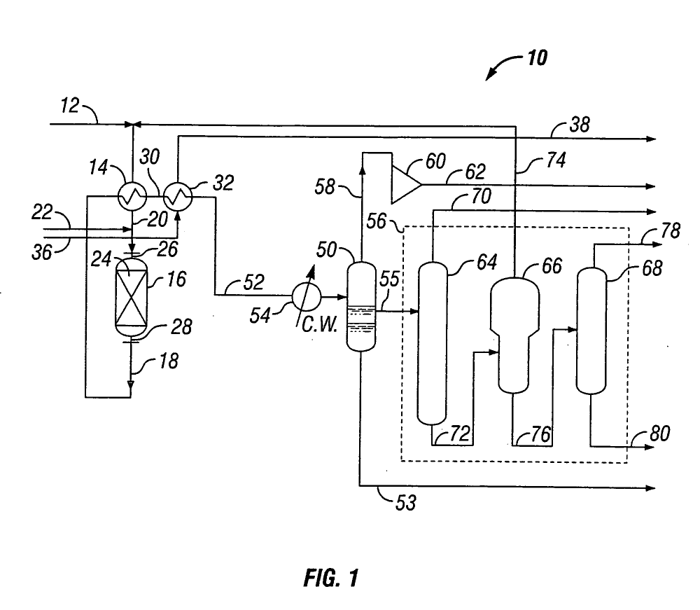

FIG. 1 presents a simplified process flow schematic of a process system for the dehydrogenation of an ethylbenzene feedstock to yield a styrene end-product, which such process system may be modified to include a high stability dehydrogenation catalyst.

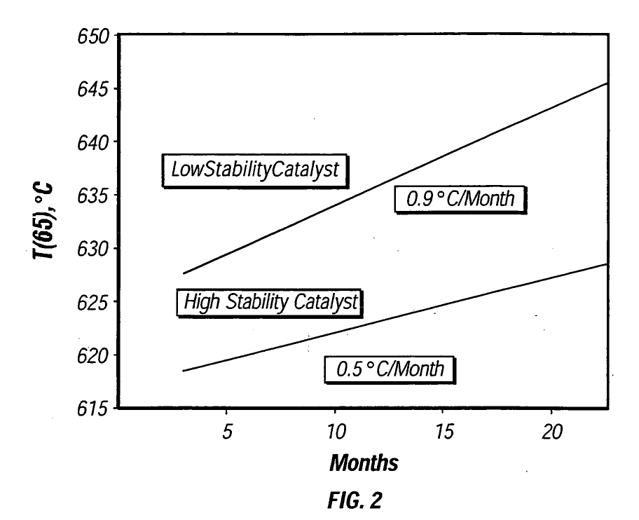

FIG. 2 presents comparative plots representative of the approximate rate of deactivation of a high stability dehydrogenation catalyst and a lower stability dehydrogenation catalyst as reflected by actual process performance data of the temperature required for a 65 percent conversion versus time in use for each catalyst.

Other objects and advantages of the invention will become apparent from the following detailed description and appended claims.

With the greater availability of high stability dehydrogenation catalysts it is becoming increasingly desirable to develop novel methods that allow for the maximization of the advantages that such high stability dehydrogenation catalysts can provide, but heretofore not yet captured, in the operation of existing dehydrogenation process systems, such as, process systems for the manufacture of styrene by the dehydrogenation of ethylbenzene. It is further desirable to develop novel methods for the design of dehydrogenation systems that maximize the advantages that are obtainable from the use of high stability dehydrogenation catalysts.

As it is used in this specification, the term stability refers to the rate at which a particular catalyst deactivates expressed in terms of the ratio of the change in catalyst activity for a given time period of use of the catalyst at specific reaction conditions (Δ activity per Δ time). It is recognized that the rate at which a catalyst deactivates may depend upon the severity of the reaction conditions at which the catalyst is utilized. In the case of an ethylbenzene dehydrogenation catalyst, i.e., a styrene manufacturing catalyst, the stability value is the ratio of the change in the activity of the styrene manufacturing catalyst, when used under certain process conditions, to a period of time in use. The stability value of a styrene manufacturing catalyst can vary depending upon the severity of the process conditions, which can include such process parameters as steam-to-oil ratio, liquid hourly space velocity, pressure and reactor temperature.

References herein to the activity of a catalyst are meant to refer to the temperature parameter associated with the particular catalyst. In the case of a styrene manufacturing catalyst, its temperature parameter is the temperature, in ° C., at which the styrene manufacturing catalyst provides under certain defined process conditions a specified conversion of an ethylbenzene feed. An illustrative example of activity is the temperature at which a conversion of 65 mole % of the ethylbenzene is achieved when contacted with the styrene manufacturing catalyst under certain specified reaction conditions. Such a temperature parameter may be represented by the symbol “T(65)”, which means that the given temperature provides for a conversion of 65 mole percent. The T(65) temperature value represents the activity of the associated catalyst. The activity of a catalyst is inversely related to the temperature parameter with higher activities being represented by lower temperature parameters and lower activities being represented by higher temperature parameters.

As used herein, the term “conversion” means the fraction, in mole %, of a specified compound converted to another compound. As an example, in an ethylbenzene dehydrogenation process, the ethylbenzene of the feedstock is considered to be the specified compound that is to be converted to another compound, such as, to benzene, toluene, styrene or other compounds.

As used herein, the term “selectivity” means the fraction, in mole %, of the converted compound that yields the desired compound. As an example, in an ethylbenzene dehydrogenation process, the ethylbenzene of the feedstock is considered to be the converted compound and the desired compound is considered to be styrene.

One aspect of the inventive method is that it provides for the improvement in the operation of an existing dehydrogenation reactor system and, in particular, in the operation of a dehydrogenation reactor system used for the dehydrogenation of ethylbenzene to yield a styrene product. A typical dehydrogenation process system includes a reaction section and a separation section. The reaction section provides for the contacting of a feedstock, which may comprise ethylbenzene, with a dehydrogenation catalyst under dehydrogenation conditions to yield a reaction section reaction product. The separation section provides for the separation of the reaction section reaction product into various of its products, such as styrene, and recycle streams, such as unconverted ethylbenzene.

The reaction section, in general, includes a dehydrogenation reactor system comprising a dehydrogenation reactor that contains a first volume of a dehydrogenation catalyst. The dehydrogenation reactor is typically a reactor vessel that defines a dehydrogenation reaction zone that contains the dehydrogenation catalyst. The dehydrogenation catalyst exhibits, or may be characterized as having, certain stability characteristics that make it less stable than high stability dehydrogenation catalysts.

The lower stability characteristics of the dehydrogenation catalyst, as compared to those of high stability dehydrogenation catalysts, can impact how the dehydrogenation reactor system is operated; since, in the operation of the dehydrogenation reactor system the dehydrogenation reaction temperature is typically raised to offset the effects of catalyst deactivation. In this method of operating the dehydrogenation reactor system, as the dehydrogenation catalyst ages and deactivates with use the dehydrogenation reaction temperature is raised until it reaches an upper temperature that is limited by the dehydrogenation process equipment or by economic considerations. When this temperature limit is reached, the dehydrogenation reactor system is considered to be operating at end-of-run conditions, at which time, the dehydrogenation reactor system is shut down and the deactivated dehydrogenation catalyst is replaced with fresh catalyst. Because the fresh catalyst is more active than the used catalyst, when the dehydrogenation reactor system is started back up, the start-of-run temperature required to achieve a given conversion of the feedstock is substantially lower than the end-of-run temperature required to achieve the same conversion.

In the typical existing dehydrogenation reactor system the reactor volume is fixed. Due to this fixed reactor volume, the replacement of a previously used or deactivated dehydrogenation catalyst with a high stability dehydrogenation catalyst will provide for the ability to either operate the dehydrogenation reactor system for a longer time period before it reaches end-of-run operating conditions, or operate the dehydrogenation reactor system at higher reactor temperatures in order to take advantage of higher conversions, or a combination of the two operating modes. The inventive method takes advantage of the stability characteristics of high stability dehydrogenation catalysts in such a way as to improve the operation of a dehydrogenation reactor system.

The inventive method of improved operation of a dehydrogenation reactor system includes removing from the dehydrogenation reactor at least a portion of the first volume of dehydrogenation catalyst, which has been used and has become at least partially deactivated through such use. Preferably, a major portion of the first volume of dehydrogenation catalyst, and, most preferably, the entire or essentially the entire first volume of the dehydrogenation catalyst is removed from the dehydrogenation reactor.

After the removal of the deactivated dehydrogenation catalyst from the dehydrogenation reactor, a second volume of a high stability dehydrogenation catalyst is placed into the dehydrogenation reactor, which is empty, or partially empty, as a result of the removal of the deactivated dehydrogenation catalyst that has become deactivated or, preferably, spent, due to its use to thereby form a second dehydrogenation reactor system having a second volume of a high stability dehydrogenation catalyst. This second dehydrogenation reactor system is then operated under suitable dehydrogenation reaction conditions.

Because of the greater stability of the replacement high stability dehydrogenation catalyst there is more flexibility in the manner by which the second dehydrogenation reactor system may be operated. To exploit this flexibility the operating conditions of the dehydrogenation reactor system are controlled so as to provide a desired deactivation rate of the high stability dehydrogenation catalyst that provides a run length that approximates a desired run length from the start-of-run to end-of-run.

The start-of-run of a dehydrogenation reactor system is typically considered to be the point in time on which the dehydrogenation reactor system containing a load of new or fresh catalyst is started up with the introduction of feed and operation at dehydrogenation reaction conditions. As earlier noted, fresh catalyst typically is more active than used fresh catalyst, and, usually it requires a lower inlet feed temperature to achieve a given conversion than does used fresh catalyst. As the fresh catalyst is used, it becomes deactivated resulting in the need to raise the inlet feed temperature to provide the same given conversion. With time, the inlet feed temperature must be raised to the point at which the dehydrogenation reactor system may not be operated due to equipment limitations or economic considerations, at which point, the dehydrogenation reactor system has reached end-of-run conditions and is shut down. The used or spent fresh catalyst is removed from the dehydrogenation reactor system to be replaced with a new load of new or fresh catalyst.

The typical run length for a dehydrogenation reactor system from start-of-run to end-of-run is in the range upwardly to about 72 or even 96 months. It is recognized that long run lengths are desired, but, typically, the duration of the run length can be limited by a variety of factors including the need for equipment maintenance and dehydrogenation catalyst performance characteristics. Taking the factors into account, a desired run length can be in the range of from about 6 months to about 60 months. More typically, the desired run length is in the range of from 8 months to 48 months, and, most typically, from 12 months to 36 months.

The dehydrogenation reactor conditions that can impact the deactivation rate of the high stability catalyst include the steam-to-oil ratio of feed charged to the dehydrogenation reactor, the inlet feed temperature, the dehydrogenation reactor pressure and the liquid hourly space velocity. The preferred approach to providing for a desired deactivation rate of the high stability dehydrogenation catalyst of the second dehydrogenation reactor system is to adjust the inlet feed temperature while maintaining the feed rate, which sets the liquid hourly space velocity. With all other parameters being constant, an increase in the inlet feed temperature will increase the rate of catalyst deactivation and a decrease in the inlet feed temperature will decrease the rate of catalyst deactivation. Adjustments in the steam-to-oil ratio may also impact the stability or rate of catalyst deactivation, but normally it is desirable to maintain the steam-to-oil ratio within a certain narrow range. The feed rate can also impact the rate of catalyst deactivation, but making adjustments in the feed rate to alter the catalyst deactivation rate is not generally desirable.

By increasing the inlet feed temperature both the feed conversion and the rate of catalyst deactivation are increased. The inlet feed temperature then can be controlled to give a desired rate of deactivation of the high stability dehydrogenation catalyst that will allow for the operation of the second dehydrogenation reactor system for a desired period of time, or run length, before the high stability dehydrogenation catalyst must be removed from the second dehydrogenation reactor system and replaced due to its deactivation.

The inlet feed temperature to the second dehydrogenation reactor system typically can be in the range of from about 500° C. to about 700° C. While the use of the high stability dehydrogenation catalyst will allow for the operation of the second dehydrogenation reactor system at a lower temperature, one of the features of the inventive methods herein is the ability to increase the reaction temperature of the second dehydrogenation reactor system so as to increase conversion, but without causing an excessive rate of catalyst deactivation that results in an early or premature shutdown of the second dehydrogenation reactor system. The upper limit of the dehydrogenation reactor inlet temperature is, generally, determined by the equipment limitations and is more typically no greater than about 700° C., and, most typically, no greater than 650° C. The lower limit on the dehydrogenation reactor inlet temperature is usually set by economic considerations; since, the lower temperatures result in reduced conversion. Therefore, more typically, the dehydrogenation reactor inlet temperature of the inventive method can be in the range of from 550° C. to 700° C., and, most typically, from 600° C. to 650° C.

The feed charged to the second dehydrogenation reactor system includes a dehydrogenatable hydrocarbon such as an alkylaromatic compound, which can include an alkyl substituted benzene compound. Among the alkylaromatic compounds, ethylbenzene is preferred. Also, it is preferred to include water as an additional component of the feed charged to the second dehydrogenation reactor system. It is preferred for the water to be in the form of steam, which provides a source of heat energy required for the dehydrogenation reaction, and its presence in the reaction zone tends to inhibit the rate of coke deposition on the dehydrogenation catalyst and thereby the catalyst deactivation rate.

One of the features of the inventive method is that the second dehydrogenation reactor system can be operated with lower steam-to-oil ratios than the alternative dehydrogenation reactor system that contains the dehydrogenation catalyst not having the high stability characteristics of the high stability dehydrogenation catalyst. The steam-to-oil ratio of the feed thus can be in the range of from 1 to 20 moles of steam per mole of hydrocarbon. Preferably, the molar steam-to-oil ratio of the feed is in the range of from 2 to 15, and, most preferably, from 4 to 12. The term steam-to-oil ratio is defined as the ratio of the total moles of steam charged to a dehydrogenation reaction zone to the total moles of hydrocarbon, e.g., ethylbenzene, charged to the same dehydrogenation reaction zone.

It is generally desirable to operate the second dehydrogenation reactor system at as low a pressure as is feasible. Thus, the reaction pressure is relatively low and ranges from vacuum pressure, such as 5 kPa (0.7 psia), upwardly to about 200 kPa (29 psi). Typically, the reaction pressure can be in the range of from 10 kPa (1.45 psia) to 200 kPa (29 psi), and, more typically, it is in the range of from 20 kPa (2.9 psia) to 200 kPa.

The liquid hourly space velocity (LHSV) can be in the range of from about 0.01 hr⁻¹ to about 10 hr⁻¹, and preferably, from 0.1 hr⁻¹ to 2 hr⁻¹. As used herein, the term “liquid hourly space velocity” is defined as the liquid volumetric flow rate of the dehydrogenation feed, for example, ethylbenzene, measured at normal conditions (i.e., 0° C. and 1 bar absolute), divided by the volume of the catalyst bed, or the total volume of catalyst beds if there are two or more catalyst beds.

The dehydrogenation catalysts contemplated herein may be any suitable catalyst composition that provides for the dehydrogenation of hydrocarbons. An example of a dehydrogenation catalyst composition includes those catalysts that comprise iron oxide, such as the iron oxide-based dehydrogenation catalysts used in the dehydrogenation of an ethylbenzene feedstock to yield a styrene product. The typical dehydrogenation catalyst compositions considered herein are the iron oxide-based ethylbenzene dehydrogenation catalysts used for the manufacture of styrene. A more typical iron oxide-based dehydrogenation catalyst comprises iron oxide and potassium oxide.

The iron oxide of the iron oxide based dehydrogenation catalyst may be in a variety of forms including any one or more of the iron oxides, such as, for example, yellow iron oxide (goethite, FeOOH), black iron oxide (magnetite, Fe₃O₄), and red iron oxide (hematite, Fe₂O₃), including synthetic hematite or regenerated iron oxide, or it may be combined with potassium oxide to form potassium ferrite (K₂Fe₂O₄), or it may be combined with potassium oxide to form one or more of the phases containing both iron and potassium as represented by the formula (K₂O)ₓ.(Fe₂O₃)_{y}.

Typical iron oxide based dehydrogenation catalysts comprise from 10 to 100 weight percent iron, calculated as Fe₂O₃, and up to 40 weight percent potassium, calculated as K₂O. The iron oxide based dehydrogenation catalyst may further comprise one or more promoter metals that are usually in the form of an oxide. These promoter metals may be selected from the group consisting of Sc, Y, La, Mo, W, Ce, Rb, Ca, Mg, V, Cr, Co, Ni, Mn, Cu, Zn, Cd, Al, Sn, Bi, rare earths and mixtures of any two or more thereof. Among the promoter metals, preferred are those selected from the group consisting of Ca, Mg, Mo, W, Ce, La, Cu, Cr, V and mixtures of two or more thereof. Most preferred are Ca, Mg, W, Mo, and Ce.

A more typical iron oxide based dehydrogenation catalyst comprises from 40 to 90 weight percent iron, calculated as Fe₂O₃, and from 5 to 30 weight percent potassium, calculated as K₂O; and, it further can comprise from 2 to 20 weight percent cerium, calculated as Ce₂O₃; and, it further can comprise from 1 to 10 weight percent molybdenum, calculated as MoO₃; and, it further can comprise from 1 to 10 weight percent an alkaline earth metal, calculated as an oxide.

Descriptions of typical iron oxide-based dehydrogenation catalysts that are used as dehydrogenation catalysts may be found in patent publications that include U.S. Patent Publication No. 2003/0144566 A1; U.S. Pat. No. 5,689,023; U.S. Pat. No. 5,376,613; U.S. Pat. No. 4,804,799; U.S. Pat. No. 4,758,543; U.S. Pat. No. 6,551,958 B1; and EP 0,794,004 B1, all of such patent publications are incorporated herein by reference.

The iron oxide based catalyst is prepared by any method known to those skilled in the art. The iron oxide based dehydrogenation catalyst comprising potassium oxide and iron oxide can, in general, be prepared by combining the components of an iron-containing compound and a potassium-containing compound, shaping these components to form particles, and calcining the particles. The promoter metal-containing compounds may also be combined with the iron-containing and potassium-containing components.

The catalyst components can be formed into particles such as extrudates, pellets, tablets, spheres, pills, saddles, trilobes, tetralobes and the like. One preferred method of making the iron based dehydrogenation catalyst is to mix together the catalyst components with water or a plasticizer, or both, and forming an extrudable paste from which extrudates are formed. The extrudates are then dried and calcined. The calcination is preferably done in an oxidizing atmosphere, such as air, and at temperatures upwardly to 1200° C., but preferably from 500° C. to 1100° C., and, most preferably, from 700° C. to 1050° C.

The high stability dehydrogenation catalysts of the inventive methods are distinguished from other dehydrogenation catalysts principally by their stability characteristics rather than by their composition. Their high stability characteristics in comparison to the other dehydrogenation catalysts, however, may be, but are not required to be, due to compositional differences. The preferred high stability dehydrogenation catalysts of the inventive methods are iron oxide-based styrene manufacturing catalysts.

As used in this specification, when referring to a “high stability” dehydrogenation catalyst, what is meant is that it will exhibit when used under certain specified standard reaction conditions a deactivation rate that averages less than 0.65° C. per 30 day time period, preferably, less than 0.6° C. per 30 day time period, and, most preferably, less than 0.5° C. per 30 day time period. The standard reaction conditions for determining the stability value of a high stability dehydrogenation catalyst for use in styrene manufacturing are when a feed mixture of ethylbenzene and steam having a molar ratio of steam-to-ethylbenzene of about 7:1 is passed over a volume of the high stability dehydrogenation catalyst contained in a reactor at a rate that provides for a liquid hourly space velocity of about 1 hr⁻¹. The temperature of the feed mixture introduced into the reactor is adjusted to provide the conversion of the ethylbenzene of 65 percent. The stability value is determined by the average increase in the feed mixture temperature necessary to maintain a constant ethylbenzene conversion of 65 percent during the time period. The stability value is expressed as the change in T(65) per change in time (30 days), (e.g., ΔT(65)/Δtime), or ° C./30 days.

The dehydrogenation catalysts contemplated herein that are not considered to be of the type having high stability will not exhibit the stability characteristics of the high stability dehydrogenation catalysts, and, generally, will exhibit stability values that are larger than those of the high stability dehydrogenation catalysts. It is understood that a larger stability value means that the catalyst will tend to deactivate with use at a greater rate than will a catalyst having a lower stability value, thus, being less stable. Therefore, the dehydrogenation catalysts that are not of the high stability type can exhibit stability values greater than 0.65° C. per 30 day period, but more typically, their stability value is greater than 0.7° C. per 30 day period, and, most typically, the stability value is greater than 0.8° C. per 30 day period.

Another of the inventions herein provides for a method of designing a dehydrogenation reactor system. This method utilizes information related to the unique stability properties of high stability dehydrogenation catalysts to provide for improved dehydrogenation reactor system designs that include a reactor that defines a reaction zone and contains a volume of a high stability dehydrogenation catalyst. It, thus, is an important aspect of the inventive design methodology to be able to characterize the high stability dehydrogenation catalyst by a catalyst stability property function which is predictive of the deactivation rate of the high stability dehydrogenation catalyst as a function of one or more standard operations conditions, process variables, or process parameters. Such a standard reactor operating condition can included, for example, the reactor feed inlet temperature, the reactor feed steam-to-oil ratio, the reactor pressure, the liquid hourly space velocity, or any combination of two or more thereof. With the knowledge of the stability characteristics of the high stability dehydrogenation catalyst, a deactivation rate required to provide for a desired run length can be predicted based on the use of the catalyst under one or more of the standard operating conditions. Once the standard reactor operating condition is determined, then the reactor volume required to provide for the desired run length is calculated or determined by applying the knowledge of the operating condition that provides for the desired run length.

In another embodiment of the design methodology, desired process parameters under which the dehydrogenation reactor system is to be operated are selected and used in determining the reactor volume. These process parameters can include a desired conversion and a desired feed rate to the reactor containing the high stability dehydrogenation catalyst. These process parameters influence the rate at which a dehydrogenation catalyst is deactivated. So, based on the specific process parameters selected, an estimate of the deactivation rate of the high stability dehydrogenation catalyst can be determined. It is recognized that the stability of the high stability dehydrogenation catalyst depends upon the particular process conditions under which it is used and that, for example, the catalyst used under high conversion conditions will have a lower stability than when it is used under lower conversion conditions. But, in any event, because the high stability dehydrogenation catalyst is more stable than other dehydrogenation catalysts its rate of deactivation will be comparatively lower when used under similar process conditions.

It is usually desirable in the design of a new dehydrogenation process system to provide for the ability to operate the dehydrogenation system between start-of-run to end-of-run for time periods that minimize excessive and uneconomical periods of downtime during which the dehydrogenation system is not in use. One consideration that is used to determine an appropriate run time can include the period of time between the start-up of the dehydrogenation system and the shutdown of the dehydrogenation system for the performance of normal or routine maintenance. Other considerations can include investment and operating costs associated with the provision of process equipment large enough to contain the necessary catalyst to operate for a desired time period. One aspect of the inventive design method is that it provides means for utilizing information concerning high stability dehydrogenation catalyst to design new more economical dehydrogenation process systems. The new designs developed by using the inventive design method can have significantly smaller reactor vessels but still provide for comparable run lengths. The smaller reactor vessels translate into lower capital investment per unit of process capacity and lower operating costs due to smaller catalyst volume requirements.

In designing a new dehydrogenation reactor system using the novel method, a desired run length for the dehydrogenation reactor system is selected. Typically, as previously noted, the run length of a dehydrogenation reactor system is influenced by a variety of factors, including, the performance properties of the catalyst used. Run lengths for a dehydrogenation process system can be in the range upwardly to about 6 or even 8 years. But, typically, the run length is in the range of from about 6 months to about 5 years, and, more typically, the run length is in the range of from 8 months to 4 years. Most typically, it is desirable for a dehydrogenation process system to have a run length between 12 months to 60 months. When referring to the run length of a dehydrogenation process system what is meant is the time that runs between when the unit is first started up with fresh catalyst to when it reaches end-of-run conditions that necessitate the shutdown of the unit to remove the deactivated catalyst.

In one step of the inventive design method, a desired run length for the dehydrogenation process system is selected. Once the catalyst stability property is determined and the desired run length is selected, a reactor volume required for the selected feed rate is determined for the new dehydrogenation process system. The new dehydrogenation process system can then be equipped with a reactor having the reactor volume, as determined by the methodology, that contains a volume of high stability dehydrogenation catalyst thereby providing a dehydrogenation reactor system comprising a dehydrogenation reactor that defines a dehydrogenation reaction zone and containing a volume of high stability dehydrogenation catalyst.

In the inventive methods herein, it is generally desirable for the dehydrogenation conversions of the feeds processed to be suitably high to make the relevant dehydrogenation processes economical. Typically, in a styrene manufacturing process, the conversion of ethylbenzene can be in the range of from about 40 percent to about 95 percent. But, more typically, the desired conversion is in the range of from 60 to 95 percent. A most desired conversion is in the range exceeding 70 percent.

Now referring to FIG. 1 wherein presented is a schematic representation of a process 10 for the manufacture of styrene by the dehydrogenation of ethylbenzene in which a modified dehydrogenation reactor system contains a high stability dehydrogenation catalyst.

In process 10, an ethylbenzene feed stream, comprising ethylbenzene, passes by way of conduit 12 to feed/effluent heat exchanger 14. Feed/effluent heat exchanger 14 defines a heat transfer zone and provides means for indirect heat exchange with the dehydrogenation reactor effluent passing from dehydrogenation reactor 16 to feed/effluent heat exchanger 14 by way of conduit 18. The heated ethylbenzene feed stream passes from feed/effluent heat exchanger 14 to dehydrogenation reactor 16 through conduit 20. Prior to the introduction of the heated ethylbenzene feed stream into dehydrogenation reactor 16, superheated steam passing by way of conduit 22 is introduced into and admixed with the heated ethylbenzene feed stream to provide additional heat required for the dehydrogenation of ethylbenzene and a desired steam-to-ethylbenzene ratio.

Dehydrogenation reactor 16 defines a dehydrogenation reaction zone that contains a bed of dehydrogenation catalyst bed 24 and provides means for contacting the heated ethylbenzene feed stream, under suitable dehydrogenation reaction conditions, with the dehydrogenation catalyst bed 24. Dehydrogenation reactor 16 further includes dehydrogenation reactor feed inlet 26 and dehydrogenation reactor effluent outlet 28. Dehydrogenation reactor feed inlet 26 provides means for receiving into the dehydrogenation reactor 16 a dehydrogenation reactor feed, such as the heated ethylbenzene feed stream, and dehydrogenation reactor effluent outlet 28 provides means for discharging from the dehydrogenation reactor 16 a dehydrogenation reactor effluent, such as an ethylbenzene dehydrogenate.

While the dehydrogenation reactor 16 is depicted as a single vessel containing a single dehydrogenation catalyst bed 24, it is recognized that multiple reactors may be used that are placed in parallel arrangement or in series arrangement and further that the multiple reactors may include interstage heating as needed.

The dehydrogenation reactor 16 and dehydrogenation catalyst bed 24 together form a dehydrogenation reactor system. In the inventive method, the operation of a dehydrogenation reactor system is improved by removing the catalyst of dehydrogenation catalyst bed 24 and replacing it with a bed of high stability dehydrogenation catalyst, which allows for the adjustment of various of the process conditions. For instance, the feed temperature at the dehydrogenation reactor feed inlet 26 may be increased to improve the conversion without shortening the catalyst life below that of the dehydrogenation catalyst of bed 24 prior to its replacement with the high stability dehydrogenation catalyst. Also, the amount of steam passing through conduit 22 and combined with the ethylbenzene passing through conduit 20 may be reduced to thereby lower the steam-to-oil ratio charged to dehydrogenation reactor 16.

A cooled dehydrogenation reactor effluent passes from feed/effluent heat exchanger 14 through conduit 30 to heat transfer unit 32, which defines a heat transfer zone and provides means for the transfer of heat from the cooled dehydrogenation reactor effluent to a cooling medium to thereby further cool the dehydrogenation reactor effluent. The cooling medium passes to heat transfer unit 32 by way of conduit 36 and the heated cooling medium passes from heat transfer unit 32 by way of conduit 38.

The cooled dehydrogenation reactor effluent passes to separator 50 by way of conduit 52. Cooler 54 is interposed in conduit 52. Cooler 54 defines a heat transfer zone and provides means for removing heat energy from the cooled dehydrogenation.

Separator 50 defines a separation zone and provides means for separating the cooled dehydrogenation reactor effluent into a hydrocarbon stream, comprising hydrocarbons, such as styrene and ethylbenzene, a water stream, comprising water, and a vapor stream, comprising hydrogen. The water stream passes from separator 50 through conduit 53. The hydrocarbon stream passes from separator 50 through conduit 55 and is charged to separation system 56. Separation system 56 defines at least one separation zone and provides means for separating dehydrogenated hydrocarbons, such as styrene, from unconverted dehydrogenatable hydrocarbons, such as ethylbenzene, and other hydrocarbons.

The vapor stream passes from separator 50 through conduit 58 and is introduced into the suction inlet of compressor 60, which defines a compression zone and provides means for compressing the vapor stream. The compressed vapor stream is discharged and passes from compressor 60 through conduit 62.

Separation system 56 can further include benzene-toluene (BT) column 64, ethylbenzene recycle column 66 and styrene finisher 68. The hydrocarbon stream from separator 50 is fed by way of conduit 55 to benzene-toluene column 64, which defines a separation zone and provides means for separating the hydrocarbon stream into a benzene/toluene stream comprising benzene and toluene and a BT column bottoms stream comprising ethylbenzene and styrene. The benzene/toluene stream passes from BT column 64 through conduit 70.

The BT column bottoms stream passes from BT column 64 through conduit 72 and is charged to ethylbenzene recycle column 66. Ethylbenzene recycle column 66 defines a separation zone and provides means for separating the BT column bottoms stream into an ethylbenzene recycle stream, comprising ethylbenzene, and an ethylbenzene recycle column bottoms stream, comprising styrene. The ethylbenzene recycle stream passes from ethylbenzene recycle column 66 through conduit 74 and is combined with the ethylbenzene feed stream being charged to feed/effluent exchanger 14 via conduit 12. The ethylbenzene recycle column bottoms stream passes from ethylbenzene recycle column 66 through conduit 76 to styrene finisher 68. Styrene finisher 68 defines a separation zone and provides means for separating the ethylbenzene recycle column bottoms stream into a styrene product stream, comprising styrene, and a residue stream. The styrene product stream passes from styrene finisher 68 through conduit 78 and the residue stream passes through conduit 80.

The following Example is presented to illustrate the invention, but it should not be construed as limiting the scope of the invention.

### EXAMPLE

This Example describes the data that is summarized in the plots of FIG. 2 for the operation of a dehydrogenation reaction systems using either a dehydrogenation catalyst that does not have high stability characteristics or a high stability dehydrogenation catalyst.

Presented in FIG. 2 are fitted plots of actual performance data of a dehydrogenation reactor system one of which contains a non-high stability dehydrogenation catalyst and the other of which contains a high stability dehydrogenation catalyst. Presented on the Y-axis is the average reactor inlet temperature normalized to a 65 percent conversion and on the X-axis is the time in months since the catalyst was first placed in service. The normalized conversion is based on the process conditions using a molar steam-to-oil ratio of about 9, a LHSV of about 0.45 hr⁻¹ and an average pressure of about 9 psia.

It is recognized that fresh styrene manufacturing catalyst needs a break-in period prior to it reaching its peak performance. This break-in or induction period is shown in FIG. 2 to be approximately three months. The data obtained for the time period subsequent to the break-in period are fitted to lines that approximate the linear rate of deactivation of the relevant catalyst. As is shown, the slope of the line representing the deactivation rate of the non-high stability dehydrogenation catalyst is greater than the slope of the line representing the high activity dehydrogenation catalyst. The non-high stability dehydrogenation catalyst shows a rate of deactivation of about 0.9° C. per month as opposed to a deactivation rate of about 0.5° C. per month for the high stability catalyst.

Reasonable variations, modifications and adaptations of the invention may be made within the scope of the described disclosure and appended claims without departing from spirit and the scope of the invention.

That which is claimed is:

1. A method of improving the operation a dehydrogenation reactor system having a dehydrogenation reactor defining a dehydrogenation reaction zone and containing a first volume of a dehydrogenation catalyst, said method comprises:
removing from said dehydrogenation reactor at least a portion of said first volume of said dehydrogenation catalyst;
placing in said dehydrogenation reactor, having removed therefrom said at least a portion of said first volume of said dehydrogenation catalyst, a second volume of a high stability dehydrogenation catalyst to thereby provide a second dehydrogenation reactor system;
operating said second dehydrogenation reactor system under a dehydrogenation reaction condition; and
controlling said dehydrogenation reaction condition so as to provide a desired deactivation rate of said high stability dehydrogenation catalyst.

2. A method as recited in claim 1, wherein said dehydrogenation catalyst comprising an iron oxide based dehydrogenation catalyst comprising from 10 to 100 weight percent iron, calculated as Fe₂O₃ and based on the total weight of said iron oxide based dehydrogenation catalyst, and up to 40 weight percent potassium, calculated as K₂O and based on the total weight of said iron oxide based dehydrogenation catalyst.

3. A method as recited in claim 2, wherein said high stability dehydrogenation catalyst has a property such that it exhibits a high stability dehydrogenation catalyst stability value exhibiting a deactivation rate that averages, under standard reaction conditions, less than 0.65° C. per 30 day time period, and wherein said standard reaction conditions include the passing of a feed mixture of ethylbenzene and steam having a molar ratio of steam-to-hydrocarbon of about 7:1 over a volume of said high stability dehydrogenation catalyst at a rate that provides a liquid hourly space velocity of about 1 hr⁻¹, and wherein said deactivation rate is defined as the ratio of change in T(65) per change in time expressed in ° C. per day.

4. A method as recited in claim 3, wherein said dehydrogenation reaction condition includes an inlet feed temperature to said dehydrogenation reactor of said second dehydrogenation reactor system.

5. A method as recited in claim 4, wherein said controlling step includes adjusting said inlet feed temperature to give said desired deactivation rate so as to provide a desired run length from start-of-run to end-of-run of said second dehydrogenation reactor system in the range of from about 6 months to about 60 months.

6. A method as recited in claim 4, wherein said controlling step includes selecting an upper temperature limit for said inlet feed temperature and adjusting said inlet feed temperature to give said desired deactivation rate so as to provide a desired run length from start-of-run to end-of-run of said second dehydrogenation reactor system in the range of from about 6 months to about 60 months in which said upper temperature limit for said inlet feed temperature is reached.

7. A method as recited in claim 6, wherein said upper temperature limit is less than 700° C.

8. A method as recited in claim 7, wherein said dehydrogenation catalyst exhibits a dehydrogenation catalyst stability value exceeding 0.65° C. per 30 day time period.

9. A method as recited in claim 4, wherein said controlling step includes adjusting said inlet feed temperature to give a desired conversion to thereby give said desired deactivation rate so as to provide a desired run length from start-of-run to end-of-run of said second dehydrogenation reactor system in the range of from about 12 months to about 60 months.

10. A method as recited in claim 9, wherein said desired conversion is in the range of from about 50 to about 90 percent.

11. A method, comprising:
designing a dehydrogenation reactor system, which includes a reactor that defines a reaction zone and contains a volume of a high stability dehydrogenation catalyst, wherein said high stability dehydrogenation catalyst is characterized by a catalyst stability property function, using a design method comprising:
selecting a desired run length for said dehydrogenation reactor system;
using said catalyst stability property function to determine a standard reactor operating condition required to provide said desired run length; and
using said standard reactor operating condition to determine a reactor volume for said reactor required to provide said desired run length; and, thereafter,
providing said dehydrogenation process system equipped with said reactor having said reactor volume and containing said volume of said high stability dehydrogenation catalyst.

12. A method as recited in claim 11, wherein said high stability dehydrogenation catalyst has a property such that it exhibits a stability value exhibiting a deactivation rate that averages, under standard reaction conditions, less than 0.65° C. per 30 day time period, and wherein said standard reaction conditions include the passing of a feed mixture of ethylbenzene and steam having a molar ratio of steam-to-ethylbenzene of about 7:1 over a volume of said high stability dehydrogenation catalyst at a rate that provides a liquid hourly space velocity of about 1 hr⁻¹, and wherein said deactivation rate is defined as the ratio of change in T(65) per change in time expressed in ° C. per day.

13. A method as recited in claim 12, wherein said catalyst stability property function defines the rate at which said high stability dehydrogenation catalyst deactivates when said dehydrogenation reactor system is operated at said standard reactor operating condition.

14. A method as recited in claim 13, wherein said desired run length is in the range of from about 6 months to about 60 months from start-of-run to end-of-run.

15. A method as recited in claim 14, wherein said standard reactor operating condition includes a liquid hourly space velocity.

16. A method as recited in claim 15, wherein said using step includes determining said reactor volume utilizing said liquid hourly space velocity.

17. A method as recited in claim 16, wherein said standard reactor operating condition further includes an inlet feed temperature.

18. A method as recited in claim 16, wherein said standard reactor operating condition further includes a feed steam-to-oil ratio.

19. A method as recited in claim 16, wherein said liquid hourly space velocity is in the range of from 0.01 to 10 hr⁻¹.
